(19) United States
(12) Patent Application Publication
(10) Pub. No.: US 2007/0270684 A1
(43) Pub. Date: Nov. 22, 2007

(54) Method and Arrangement Relating to Testing Objects

(75) Inventors: Peter Cawley, London (GB); Anders Pettersson, Goteborg (SE)

Correspondence Address: SYNNESTVEDT LECHNER & WOODBRIDGE LLP, P O BOX 592, 112 NASSAU STREET, PRINCETON, NJ, 08542-0592, US

(73) Assignee: Integration Diagnostics Ltd., London (GB)

(21) Appl. No.: 10/561,362

(22) PCT Filed: Jun. 21, 2004
(86) PCT No.: PCT/SE04/00998

§ 371 Date: Mar. 8, 2007

(51) Int. Cl. A61B 5/103

(52) U.S. Cl. 600/422; 600/410; 600/421

(57) ABSTRACT

The invention relates to an arrangement (100, 200, 500) and a method for testing a hold (120, 220, 520), such as an implant, attached to an object (130, 230, 530), such as a bone, the method comprising the steps of bringing a member (110, 210, 510) into contact with said hold, contactlessly detecting at least one resonance frequency of said member (110, 210, 510) when it is in contact with said hold (120, 220, 510); and interpreting the detected resonance frequency in terms of the degree of attachment of the hold with respect to the object.

### TECHNICAL FIELD

The present invention relates to a method and apparatus for testing an object, such as an implant attached to a bone of a human or animal subject.

### BACKGROUND OF THE INVENTION

The use of implants involves the insertion of a metal fixture into a prepared hole in the bone. During the healing process, the surrounding bone develops an intimate contact with the implant surface and after a suitable time a prosthesis may be attached to the fixture. Such implants are frequently used in dentistry and in cosmetic surgery.

There is a need for a means of clinically observing the quality of the union between the bone and the implant surface. Implant failures can be caused by errors in placement, and premature or inappropriate loading. A non-destructive test, which could be used before loading the implant would help to reduce failures of this type, and would also enable periodic tests to be carried out on implants which are in use to ensure that they are still satisfactory. The test could also provide a quantitative comparison between different implant systems.

X-rays are sometimes used to test the condition of an implant, but they can only show the presence of gross bone loss around the implant. It is also very difficult to monitor the progress of integration over time with x-rays, since it is difficult to reproduce the viewing position and angle with sufficient accuracy. A different sort of test, albeit a crude one, is to tap the structure attached to the implant with a surgical instrument. This test can only distinguish between satisfactory implants and the most grossly defective systems.

The international patent application no. WO 92/18053, relates to a method of testing an implant attached to a bone of a human or animal subject. The method comprises the steps of bringing a member into contact with the implant; detecting at least one resonance frequency of the member when it is in contact with the implant; and interpreting the detected resonance frequency in terms of the degree of attachment of the implant with respect to the bone. However, the method implies using an analysing unit being in contact with the implant through a wire.

U.S. Pat. No. 3,355,933 and WO 99/46603 relate to measuring arrangements for measuring surface vibrations of a large object. The arrangements presented in these documents are not suitable for small spaces, such as a mouth of a patient.

### SUMMARY OF THE INVENTION

The main object of the present invention is to provide contactless testing apparatus and method of an object, especially an object in a small space such as an implant.

Another object of the present invention to provide a non-destructive test, which is capable of giving a reliable indication of the quality and/or extent of the union between an implant and the bone to which it is attached in a contactless way.

Another object of the present invention is to provide a testing arrangement, which is disposable providing a purity aspect to the invention.

For these reasons, a method and arrangement for testing an implant attached to an object, such as a bone, are provided. The method comprises the steps of: bringing a member into contact with the implant, contactlessly detecting at least one resonance frequency of the member when it is in contact with the implant and interpreting the detected resonance frequency in terms of the degree of attachment of the implant with respect to the bone. The method further comprises the step of detachably attaching the member to the implant. Preferably the member comprises a cantilever beam.

According to most preferred embodiment of the invention the member comprises a magnetic part. Thus, the resonance frequency is detected by means of a coil.

According to another embodiment, the member comprises a marking. Thus, the resonance frequency is detected by means of an illumination detector.

According to another embodiment of the invention, the member is made of a ferromagnetic material. Thus, the resonance frequency is detected by means of the member disturbing a magnetic field.

Preferably, the implant includes a threaded bore, and the cantilever beam is screwed to or into the implant.

The method further comprises the step of comparing the detected resonance frequency with one or more values for the resonance frequencies of the same or similar members in contact with other implants. In one step, the detected resonance frequency is compared with one or more values, taken at different times, for the resonance frequencies of the same or similar member in contact with the same implant.

The method further includes the steps of exciting the member with a force, detecting the response of the member to the force and deriving an output, which is the ratio of the voltage of the response signal to that of the excitation signal.

The invention also relates to an arrangement for testing an implant attached to a bone. The arrangement comprises: a member adapted to be releasable attached to the implant, and detecting means for detecting at least one resonance frequency of the member when it is attached to the implant. The member comprises a detectable part. The detecting means comprises a detector for contactless detection of the detectable part.

According to most preferred embodiment, the detectable part comprises a magnetic member. Thus, the detector comprises a coil. The arrangement further comprises an amplifier, a processor, and a data store. The signals detected by the detector are amplified by the amplifier and applied as an input to be analysed. The analysed output, which represents a ratio of a response voltage to the excitation, is fed to the processor, which varies the frequency output of the oscillator of the analyser, and stores the results in the data store.

According to another aspect of the invention, the detectable part comprises a marker. Thus, the detector comprises an illumination detector and an illuminator.

According to another aspect of the invention, the detectable part consists of a ferromagnetic material.

According to another aspect of the invention, the detector comprises a coil for detecting disturbances in an external magnetic field.

Most preferably the member comprises a cantilever beam. Advantageously, the beam is arranged or adapted to resonate at a frequency within the range of about 1 to 20 kHz, preferably about 1 to 10 kHz, and more preferably of the order of about 8 kHz.

For sanitary reasons the member is disposable.

The invention also relates to a disposable implant testing part provided for testing an implant attached to a bone. The disposable implant testing part comprises a detectable part, which can be detected contactless by means of a detector.

### SHORT DESCRIPTION OF THE DRAWINGS

The invention will now be further described, by way of example only, with reference to the accompanying drawings, in which:

FIG. 1 is a schematic cross-sectional view of one embodiment of an implant testing member and apparatus according to the first aspect of the invention;

FIG. 2 is a schematic cross-sectional view of one embodiment of an implant testing member and apparatus according to the second aspect of the invention;

FIG. 3 is a graphical representation of the hypothetical change in the received amplitude with respect to the frequency of a testing beam according to the invention attached to a typical implant;

FIG. 4 is a graphical representation of the hypothetical change in the relative frequency with respect to the bone modulus of the testing beam according to the invention; and

FIG. 5 is a schematic cross-sectional view of one embodiment of an implant testing member and apparatus according to the third aspect of the invention.

### DETAILED DESCRIPTION OF THE PREFERRED EMBODIMENTS

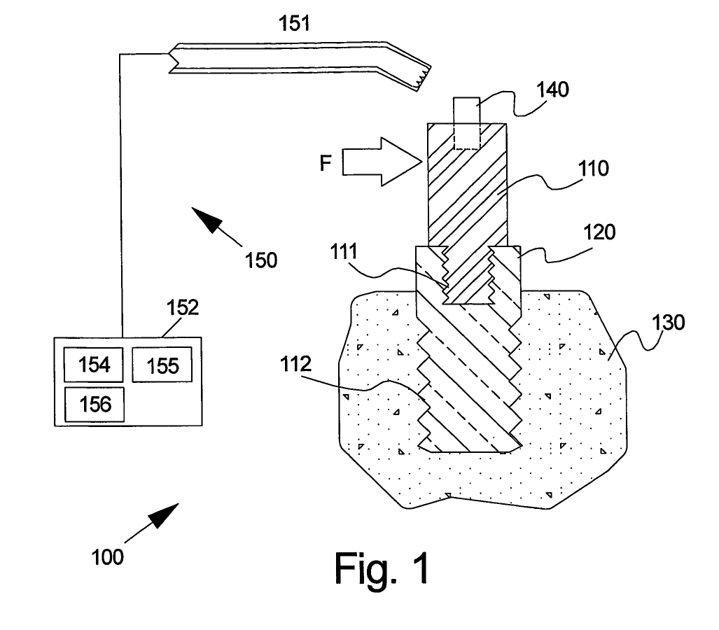

Referring to FIG. 1, the apparatus 100 comprises two parts, a member 110 in the form of a cantilever beam attached by means of a threaded section 111 to an implanted fixture 120. The implant fixture can be a dental implant attached by a threaded section 112 in a section of a bone 130, typically a human jawbone or any other type of an implant for humans or animals. The implant 120 may be any one of a number of known types, formed from a metal, such as titanium, from a ceramic material, or any other appropriate material. It may, for example, be of the type supplied by Nobel Biocare in the U.K. The member 110 is provided with a magnetic member 140. The magnetic member 140 can be provided at one end of the beam 110, e.g. the free end or integrated inside the beam.

The second part of the apparatus comprises the testing apparatus 150, including a probe 151 and a response analyzer unit 152. The probe 150 comprises a coil 153 for detecting oscillations of the magnetic member.

To generate oscillations in the beam, it must be excited. This can be done manually or by means of an electrical exciter, through application of a force F on the beam.

Signals detected by the probe 151 are amplified by an amplifier 154 and applied as an input to the analyser. The output from the analyser, which represents the ratio of the response voltage to the excitation, is fed to a processor such as a microprocessor 155, which is used to vary the frequency output of the oscillator of the analyser, and store the results in a data store 156. The results can be printed out, and/or displayed on a display or the like.

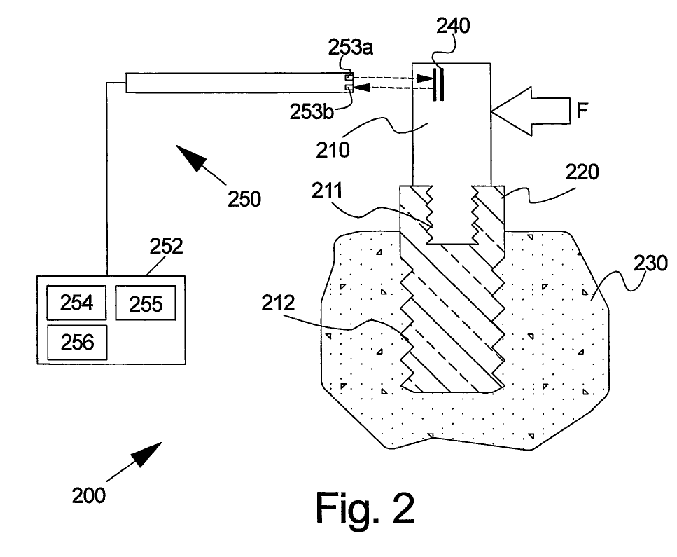

Referring now to FIG. 2, illustrating a second embodiment of the invention, the first part of the arrangement 200 according to the invention comprises, a member 210 in the form of a cantilever beam as in the earlier embodiment attached by means of a threaded section 211 to the implanted fixture 220. Also, in this case, the implant fixture can be a dental implant attached by a threaded section 212 in a section of a bone 230. The member 210 in this case is provided with markings 240, such as lines, arranged at one end of the beam 210.

The second part of the arrangement comprises the testing apparatus 250, including a probe 251 and a response analyzer unit 252. The probe 250 comprises a light source 253a, preferably but not exclusively a laser, and a light detector 253b for detecting reflections from the beam and thus oscillations of the beam. The light source is preferably Laser diode. The beam is provided with one or several markers, such as darker (or lighter) sections, which effect the reflection of the light.

The beam is excited manually or e.g. by means of an electrical exciter, by applying the force F on the beam.

The light source on the tip of the probe illuminates the beam and the light detector 253b detects the reflected light. The detected light signal is converted to an electrical signal by the detector, and signals detected by the probe 251 are amplified by an amplifier 254 and applied as an input to the analyser. The output from the analyser, which represents the ratio of the response voltage to the excitation, is fed to a processor such as a microprocessor 255, which is used to vary the frequency output of the oscillator of the analyser, and store the results in a data store 256. The results can be printed out, and/or displayed on a display or the like.

In use the beam 110 is secured, i.e. screwed, to the implanted implant 120 with a predetermined torque, for example using a torque controller and counter tool. The variations in resonance frequency with torque have been found to be relatively small over a practical range of torques, for example of the order of 5 to 10 Ncm, so that such torque variations should not present a problem.

Preferably, but not necessarily, the beam according to the invention is disposable, which means that it can be screwed off and disposed, providing a hygienic testing arrangement.

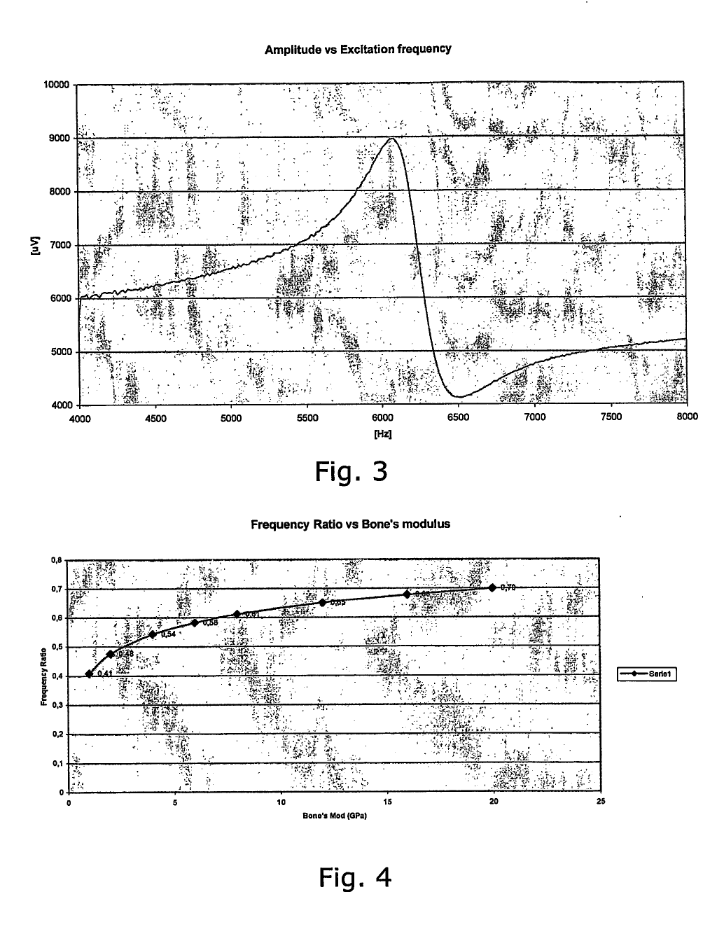

FIG. 3 shows the data from a coarse sweep, which is used to obtain the resonance frequency roughly in the apparatus of FIG. 1. A finer sweep around this region is then used to identify this frequency, typically the first or fundamental frequency, more accurately. This frequency is noted, and compared, for example, with the data for other implants at similar stages of bonding.

It is expected that for a particular implant, the resonance frequency will vary with the degree of attachment to the bone. Thus by comparing the detected resonance frequency with previously compiled data for similar implants, an indication of the degree of attachment of the implant can be obtained.

The technique, which is based on detection and comparison of resonance frequency shifts, rather than amplitude changes, is effective to determine the quality of the implant/tissue interface as a function of its stiffness, and also in relation to any bone loss as a function of the level or height of the marginal bone surrounding the implant.

The beam is preferably of a metallic material, for example titanium or aluminium, is dimensioned so as to provide a resonant frequency range of the system (placed implant and beam) of the order of 1 to 20 kHz, more specifically 1 to 10 kHz, and preferably in the region of about 8 KHz. For example, in the embodiment of FIG. 1, the upright beam can be approximately 1 cm high.

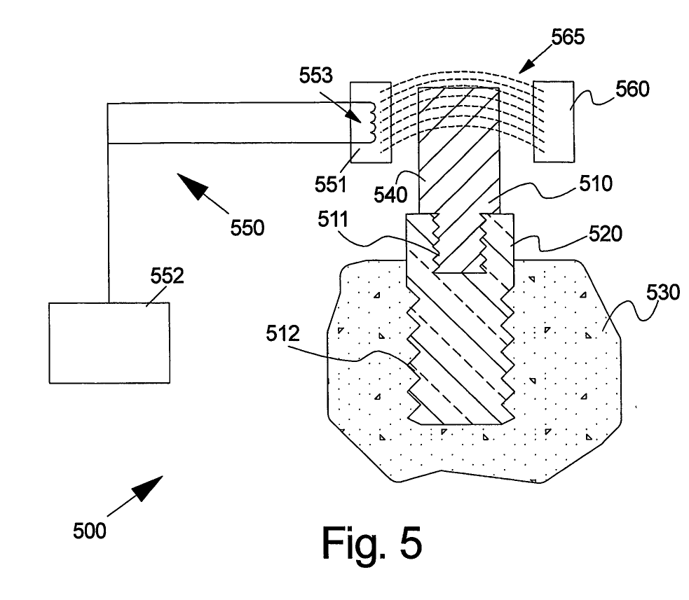

In yet another embodiment, as illustrated in FIG. 5, the first part of the arrangement 500 according to this aspect of the invention comprises a member 510 in the form of a cantilever beam made of a ferromagnetic material attached by means of the threaded section 511 to the implanted fixture 520. Also, in this case, the implant fixture can be a dental implant attached by a threaded section 512 in a section of a bone 530. Thus, the member 510 in this case is itself the detectable part 540.

The beam 510 is brought into excitation by means of an external magnetic field 565 generated by the field generators 560.

The testing apparatus 550 includes the probe 551 and the response analyzer unit 552. The probe can be part of the magnetic field generator. The probe 550 comprises a coil 553 for detecting interferences in the magnetic field 565. The analysing can be conducted as described in conjunction with the fist embodiment.

The field generator can be a permanent magnet for generating a DC field or a coil for generating an AC filed. The probe may also be externally arranged.

It will be understood that various modifications may be made without departing from the scope of the present invention as defined in the appended claims.

The transducers or gauges, and optionally also the beam may be coated, for example with an air-dry acrylic material, to protect the transducers during sterilization of the apparatus. The member may take a form other than a cantilever beam. The beam, instead of being basically straight, could be generally U-shaped, and connected to the implant or abutment by its base. Moreover, alternative detectors, such UV, sound, and the like can also be used.

The invention is not limited to implants and can be applied in preferably all small spaces wherein hold of an object such as screws, rivets, bolt or pin, is to be tested.

What is claimed is:

1. A method of testing an attachment attached to an object, the method comprising the steps of: bringing a member into contact with said attachment, bringing said member into vibration, contactlessly detecting at least one resonance frequency of said member; and interpreting the detected resonance frequency in terms of the degree of attachment of the attachment with respect to the object.

2. The method according to claim 1, including the step of detachably attaching said member to said attachment.

3. The method according to claim 1, wherein said member comprises a cantilever beam.

4. The method according to claim 1, wherein said member comprises a magnetic part.

5. The method according to claim 4, wherein said resonance frequency is detected by means of a coil.

6. The method according to claim 1, wherein said member comprises a marking.

7. The method according to claim 6, wherein said resonance frequency is detected by means of an illumination detector.

8. The method according to claim 1, wherein said member is made of a ferromagnetic material.

9. The method according to claim 8, wherein said resonance frequency is detected by means of said member disturbing a magnetic field.

10. The method according to claim 3, wherein said attachment includes a threaded bore, and said cantilever beam is screwed to or into the attachment.

11. The method according to claim 1, including the step of comparing the detected resonance frequency with one or more values for the resonance frequencies of the same or similar members in contact with other attachments.

12. A method according to claim 1, including the step of comparing the detected resonance frequency with one or more values, taken at different times, for the resonance frequencies of the same or similar member in contact with the same attachment.

13. The method according to claim 1, including the steps of exciting the member with a force, detecting the response of the member to the force.

14. A method according to claim 13, including deriving an output which is the ratio of the voltage of the response signal to that of the excitation signal.

15. A method according to claim 1, wherein said attachment is an implant and said object is a bone.

16. An arrangement for testing an implant attached to a bone, the arrangement comprising: a member adapted to be releasably attached to said implant, detecting means for detecting at least one resonance frequency of the member when it is attached to the implant, wherein said member comprises a detectable part and said detecting means comprises a detector for contactless detection of said detectable part.

17. The arrangement according to claim 16, wherein said detectable part comprises a magnetic member.

18. The arrangement according to claim 16, wherein said detector comprises a coil.

19. The arrangement according to claim 16, further comprising an amplifier, a processor, and a data storing arrangement.

20. The arrangement according to claim 16, wherein signals detected by the detector are amplified by said amplifier and applied as an input to be analysed; the analysed output, which represents a ratio of a response voltage to the excitation, is fed to said processor, which varies the frequency output of the oscillator of the analyser, and stores the results in said data storing arrangement.

21. The arrangement according to claim 16, wherein said detectable part comprises a marker.

22. The arrangement according to claim 21, wherein said detector comprises an illumination detector.

23. The arrangement according to claim 21, further comprising an illuminator.

24. The arrangement according to claim 16, wherein said detectable part consists of a ferromagnetic material.

25. The arrangement according to claim 24, wherein said detector comprises a coil for detecting disturbances in an external magnetic field.

26. The arrangement according to claim 16, wherein the member comprises a cantilever beam.

27. The arrangement according to claim 26, wherein the beam is arranged or adapted to resonate at a frequency within the range of about 1 to 20 kHz.

28. The arrangement according to claim 16, wherein said member is disposable.

29. A disposable implant testing part provided for testing an implant attached to a bone, said part comprising a detectable part, which can be detected contactless by means of a detector.

30. The arrangement according to claim 27, wherein the beam is arranged or adapted to resonate at a frequency within the range of about 1 to 10 kHz.

31. The arrangement according to claim 30, wherein the beam is arranged or adapted to resonate at a frequency of about 8 kHz.
